(19) United States
(12) Patent Application Publication
(10) Pub. No.: US 2024/0261476 A1
(43) Pub. Date: Aug. 8, 2024

(54) THIOLATED POLYMER GEL WITH INTESTINAL ABSORPTION BARRIER AND USING METHOD THEREOF

(71) Applicants: National Health Research Institutes, Zhunan Town (TW); National Taiwan University, Taipei (TW)

(72) Inventors: Feng-Huei LIN, Zhunan Town (TW); Tzu-Chien CHEN, Taipei (TW); Rui-Chian TANG, Zhunan Town (TW)

(21) Appl. No.: 18/529,540

(22) Filed: Dec. 5, 2023

(30) Foreign Application Priority Data
Feb. 8, 2023 (TW) 112104458

(51) Int. Cl. A61L 31/04; C08L 5/04

(52) U.S. Cl. CPC A61L 31/042; C08L 5/04

(57) ABSTRACT

The present invention uses the natural polymer alginate, as an example, to cross-link with Thioglycolic acid to form a sulfur polymer gel called Thiolated Alginate (TA). Thiolated alginate (TA) utilizes the redox reaction of the mucous membrane to the sulfur group, so that its materials can be covalently bonded by disulfide bonds, so as to prolong the residence time of the materials in the intestinal tract, at the same time, to form a thin film in the intestinal tract to reduce energy intake and achieve the effect of preventing or controlling obesity.

### BACKGROUND OF THE INVENTION

### Technical Field of the Invention

The present invention relates to a gel, more particularly, a thiolated polymer gel with the ability to form intestinal absorption barrier and uses thereof.

### Background

Obesity is often accompanied by numerous chronic diseases and fatal complications, such as type 2 diabetes, cardiovascular disease, coronary artery atherosclerosis, stroke, etc. The main cause of obesity is an imbalance in nutrients (such as excessive intake of nutrients or insufficient physical activity), leading to an accumulation of excess energy in the form of fat within the body. Previous techniques aimed at nutrient absorption included various surgeries. For instance, the first one is Roux-en-Y gastric bypass, which involves modifying the gastrointestinal tract to bypass a significant portion of the stomach and the duodenum, thereby reducing food absorption. The second one is a gastric balloon, which reduces the capacity of the stomach and increases satiety to decrease food intake. However, these invasive methods are only suitable for severely obese individuals, and only about one percent of patients are willing to undergo such surgeries, indicating a low overall acceptance rate.

In 2007, a less invasive surgery called “Endobarrier” was developed for patients with type 2 diabetes, aiming to control body weight by inhibiting nutrient absorption. This surgery places a device near the pylorus using an endoscope, secures it with metal rings, and uses a plastic membrane as a physical barrier to prevent contact between food and the intestinal mucosa, thereby blocking nutrient absorption in the intestines. Although Endobarrier helps to improve weight control, it requires endoscopic removal every year, and its side effects include nausea, abnormal cell tissue proliferation in the gastrointestinal tract, bleeding, and even device dislodgement. Therefore, there is a need for a weight control method that is safe, effective, and has a higher patient compliance rate.

### SUMMARY OF THE INVENTION

The present invention provides a thiolated polymer gel with intestinal absorption barrier, comprising a natural macromolecule (polymer) that has been modified through thiolation, and uses thereof creating intestinal absorption barriers, aiding in weight loss, managing diabetes, and addressing metabolic syndrome as a thiolated polymer gel.

The present invention provides a thiolated polymer gel with intestinal absorption barrier, comprising sodium alginate that is dissolved in water. Thioglycolic acid (TGA) is added to the sodium alginate solution under acidic environment at pH 2 and at a temperature range of 70-90° C., creating a mixture. The mixture is then poured into 95% ethanol, resulting in the precipitation of the mixture, which becomes thiolated alginate (TA). Thiolated alginate (TA) is derived from introducing thiol groups to modify sodium alginate. The thiol groups in thiolated alginate (TA) can undergo redox reactions with thiol groups present on the mucosal layer of the gastrointestinal system, forming reversible disulfide bonds. This allows the material to stay on the mucosal layer of the gastrointestinal system for an extended period without causing excessive accumulation in the body. Furthermore, the thiolated polymer gel can serve as a temporary physical absorption barrier between the gastrointestinal system and food nutrients to reduce the absorption of food nutrients in the gastrointestinal system.

The present invention provides a thiolated polymer gel with intestinal absorption barrier, wherein the thiolated alginate (TA) is freeze-dried and stored at 4° C. for future use.

The present invention provides a thiolated polymer gel with intestinal absorption barrier, wherein the thioglycolic acid (TGA) is added to the sodium alginate, dissolved in water under acidic conditions (pH 2) at a temperature range of 70-90° C.

The present invention provides a method of using the thiolated polymer gel with intestinal absorption barrier, which comprises administering a reactant carrier that contains the thiolated polymer gel with intestinal absorption barrier to a subject, wherein the reactant carrier is formed using thiolated alginate (TA), and can be in at least one of the following forms: chewing gum, granulated candy, granulated chocolate, granulated biscuit, pellet, tablet, capsule, powder, or gel food pouch.

The present invention provides a method of using the thiolated polymer gel with intestinal absorption barrier, wherein the thiolated polymer gel can be implemented through at least one of the following methods: endoscopic examination, needle, brush, puncture device, tube, straw, or spray.

The objective of the present invention is to develop an edible and biodegradable health supplement that forms a temporary coating on the mucosal layer of the intestine, acting as an intestinal absorption barrier. The present invention utilizes natural polymers to form thiolated polymer gels through the chemical modification of the thiol group. The thiolated polymer gel can form temporary disulfide bonds with mucins present on the mucosal layer of the intestine through the natural redox balance in the body. This not only increases the residence time of the material in the intestine but also helps to block excessive nutrient absorption.

Most weight control products rely on the addition of water-soluble fibers to induce satiety and reduce food absorption, but they still cannot effectively control nutrient absorption in the body. In contrast with other health supplements or weight control products, the present invention provides a gel that achieves a reduction in nutrient absorption through the “absorption barrier”. In one embodiment, the thiolated polymer gel after modification not only effectively blocks nutrient absorption but also increases the residence time of the gel in the intestine due to the formation of disulfide covalent bonds with the mucosal layer through thiol groups. The thiolated polymer gel can be orally administered before meals without restricting diet or changing exercise habits and still effectively reduces weight and prevents an increase in body fat in animals fed a high-fat diet.

The present invention demonstrated that the thiolated polymer gel has been proven effective in blocking glucose absorption in animal experiments. After 10-week administration, the oral glucose tolerance test (OGTT) was determined again. The results showed that consuming thiolated alginate (TA) effectively reduces insulin resistance and stabilizes blood sugar levels. The 10-week animal study demonstrated significant reductions in body weight, low-density lipoprotein in the blood, as well as the formation of visceral and subcutaneous fat and fatty liver. The raw materials of the present invention are easily obtainable, the manufacturing process is simple and does not require expensive equipment (it can be synthesized in just four hours), and it has a lower cost and does not cause cellular toxicity. The present invention can be used not only for individuals with mild obesity or overweight but also for those with metabolic syndrome and diabetes. Furthermore, in the future, it can be utilized as a sustained-release drug carrier to meet various needs based on different conditions. The thiolated alginate (TA) not only exhibited excellent biocompatibility but also demonstrated 1.65 times higher mucosal adhesion properties compared to regular alginate, as confirmed in microfluidic experiments. In the animal experiment, C57BL/6 mice were fed a high-fat diet (HFD) and evaluated for the effectiveness of the material in weight loss treatment through daily administration of thiolated alginate (TA). The mice group that received long-term TA treatment exhibited only a slight increase in body weight, reduced accumulation of body fat, improved insulin resistance, and alleviated fatty liver symptoms. Biochemical analysis of serum showed decreased levels of AST, cholesterol, and low-density lipoprotein (LDL). Therefore, the thiolated alginate (TA) can be considered one of the candidate foods for the treatment of obesity and related metabolic syndrome.

### BRIEF DESCRIPTION OF DRAWINGS

FIG. 1A shows the structural formula of alginate.

FIG. 1B shows the structural diagram of mucin.

FIG. 1C shows thiolated alginate can form short-term adhesions to the small intestine through the formation of disulfide bonds.

FIG. 1D shows the schematic representation of mucosal adhesion of thiolated alginate and mucin. (This diagram isn't original.)

FIG. 2A shows the relative fluorescence intensity of the thiolated alginate-FITC (TAF) group and the alginate-FITC (AF) group were evaluated by in vitro mucosal adhesion test.

FIG. 2B shows the glucose levels of the alginate (ALG) group and the thiolated alginate (TA) group were evaluated by in vitro barrier function test.

FIG. 2C shows images of the alginate-FITC (AF) group generated by the 3D bioluminescent imaging system IVIS.

FIG. 2D shows images of the thiolated alginate-FITC (TAF) group generated by the 3D bioluminescent imaging system IVIS.

FIG. 3 shows the line graph of blood glucose levels in the thiolated alginate (TA) group and the control group.

FIG. 4A shows the schematic representation of long-term efficacy testing of thiolated alginate (TA) in animal models.

FIG. 4B shows the line graph of weight changes in long-term efficacy testing of thiolated alginate (TA) in animal models.

FIG. 5 shows weight changes in white adipose tissue of mice on a high-fat diet (HFD) with thiolated alginate (TA) supplementation.

FIG. 6A shows the images of cell viability which were determined by live/dead staining.

FIG. 6B shows the images of visceral organs which were stained with hematoxylin and eosin (H&E) stain.

FIG. 7 shows the specific reduction in individual weight and average weight using thiolated alginate (TA) material.

FIG. 8 shows the flow chart of producing thiolated alginate (TA).

### DETAILED DESCRIPTION OF THE INVENTION

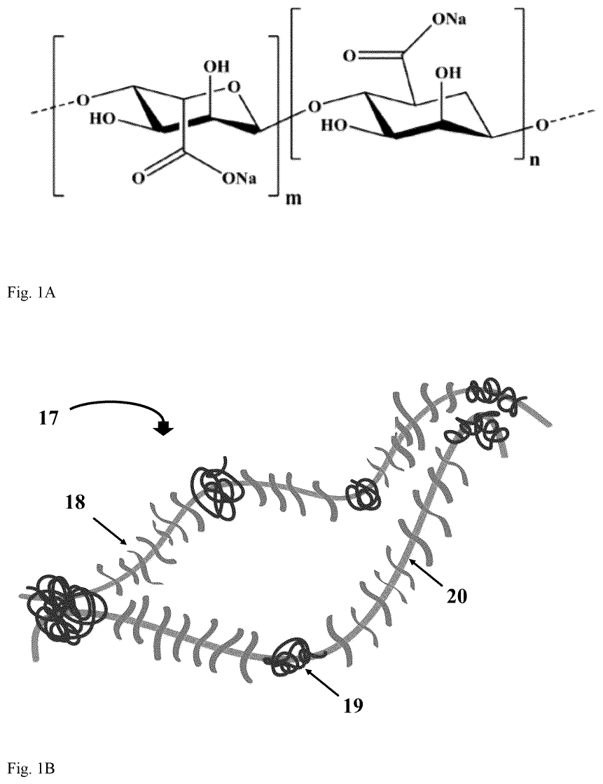

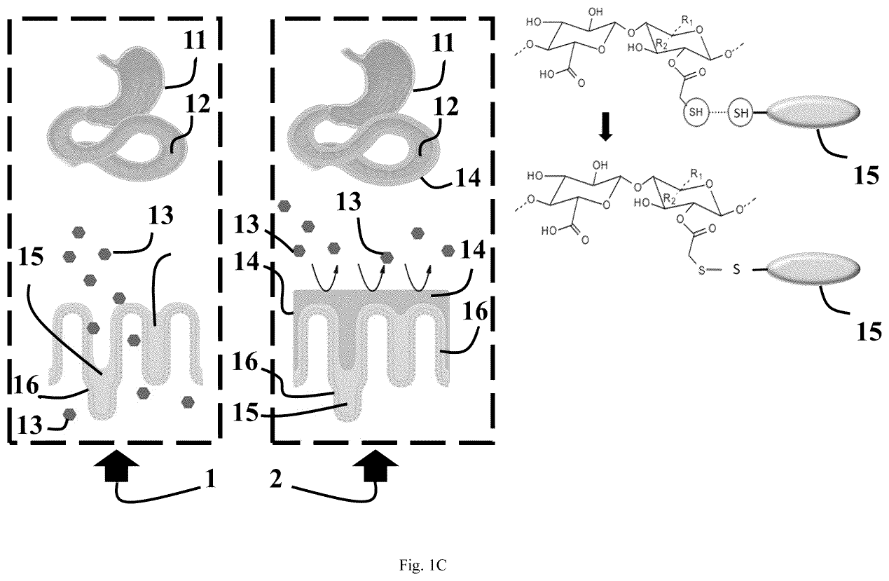

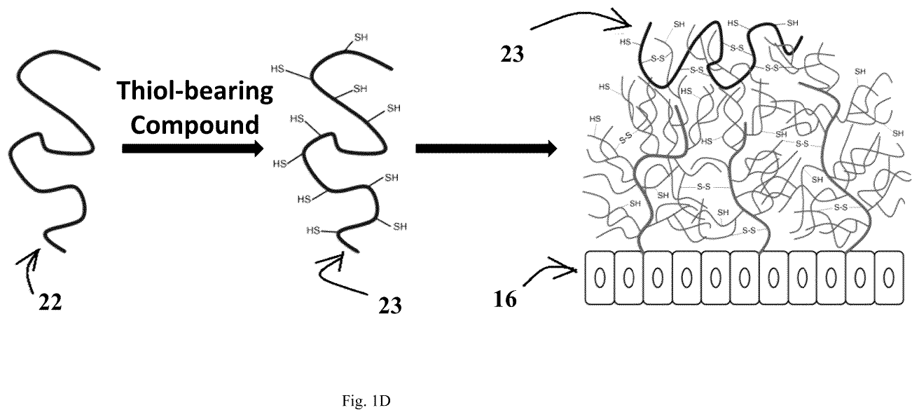

As shown in FIG. 1A, alginate is an algal compound that is a biocompatible, biodegradable and non-toxic natural polymer. Alginate is widely utilized in drug and cell delivery systems. Alginate can inhibit digestive enzymes such as a-amylase, a-glucosidase, pepsin and lipase; thus, alginate is a potential medication for obesity treatment. Due to its water solubility, alginate cannot attach to the gastrointestinal tract for a long period of time and withstand gastrointestinal peristalsis, resulting in rapid passage through the digestive system. Consequently, daily administration of alginate may not be highly effective for weight control. The present invention provides a thiolated polymer gel that specifically prolongs the retention time of alginate in the intestines. It can reduce nutrient intake and achieve anti-obesity effects by rapidly covering the mucosal layer of the gastrointestinal tract with alginate.

FIG. 1B shows the structure of mucin. In one embodiment, in the intestinal microenvironment, the intestinal mucus layer contains cysteine in certain glycoprotein domains, such as muc2, which is a major component of the mucus layer secreted by goblet cells. To regulate important physiological functions, such as redox electron transfer in the digestive system and immune response against pathogens, the mucus layer on the mucosal surface forms or breaks disulfide bonds between mucosal layers to regulate the mobility or viscosity of the mucosa.

As shown in FIGS. 1B and 1C, the mucus layer of the intestinal epithelium consists of numerous glycoproteins. Mucin is a glycoprotein with hyperglycosylation. Within the main chain of mucin, proline, threonine, and serine form a PTS structure, and many modifications are found in its side chain. Proteins that contain more than 10% cysteine tend to aggregate mucin monomers and form a three-dimensional network, while disulfide bonds break, decreasing viscosity. Based on this mechanism, the present invention provides a thiolated polymer gel with an intestinal absorption barrier that modifies alginate with thiol groups, wherein the thiolated alginate (TA) can form disulfide bonds in the intestinal tract, allowing it to stay on the mucosal layer for an extended period.

Moreover, in one embodiment shown in FIGS. 1B and 1C, the disulfide bonds formed between the materials and mucin are reversible. The thiolated alginate (TA) can form disulfide bonds through sulfhydryl groups between the materials and the mucosal layer and prolong its attachment time in the small intestine. Subsequently, the disulfide bonds are broken down, allowing the material to separate from the mucosal layer and be expelled from the body along with the food bolus, preventing accumulation in the body. Therefore, the material properties of the thiolated polymer gel with an intestinal absorption barrier, modified with sulfur groups, allow for an extended duration and action of the material on the mucosa. In the gastrointestinal tract, free sulfhydryl groups can form disulfide bonds through the redox regulation of the body. The balance of oxidation-reduction between glutathione/glutathione disulfide and cysteine/cystine can maintain the integrity of the mucosa.

As shown in FIG. 1B, the main chain of mucin consists of PTS structures, while the side chains undergo numerous glycosyl group modifications that exist in the area rich in cysteine and globular proteins. FIG. 1C shows the diagram of mucin 15, intestinal epithelial 16, mucin glycoproteins 17, oligosaccharides 18, cysteine rich domain 19, and core protein 20.

FIG. 1D shows the diagram of intestinal epithelial 16, polymer 22, and thiolated polymer 23.

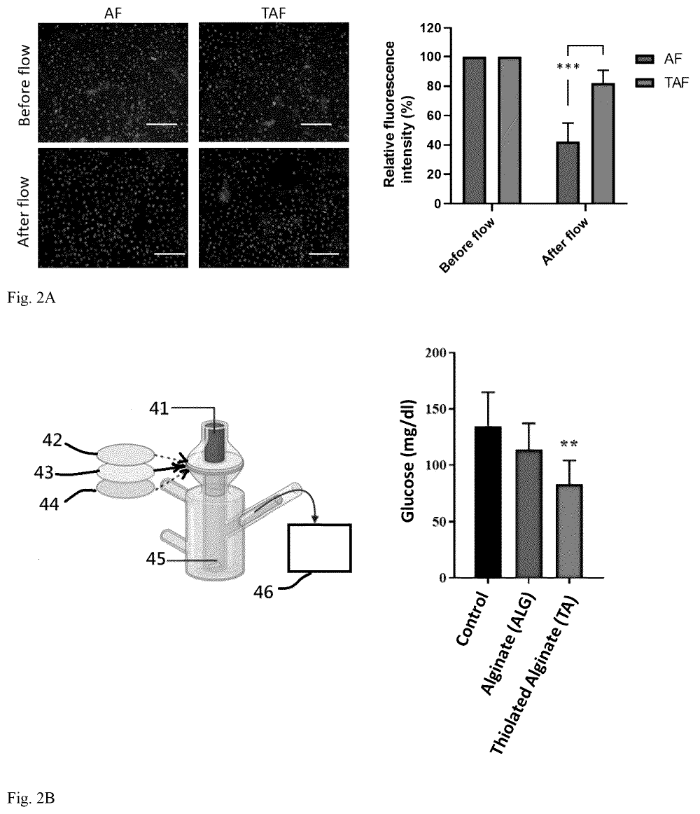

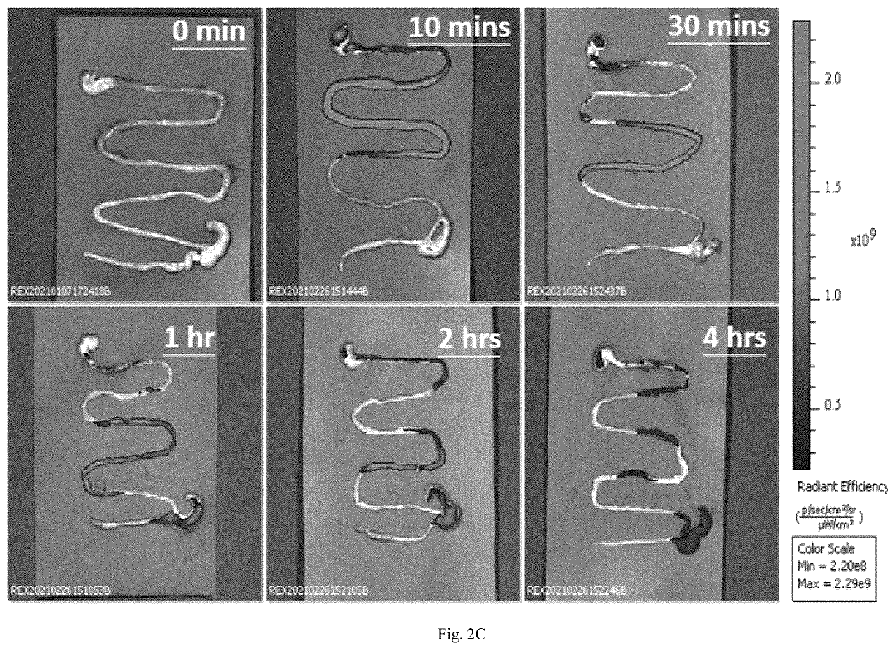

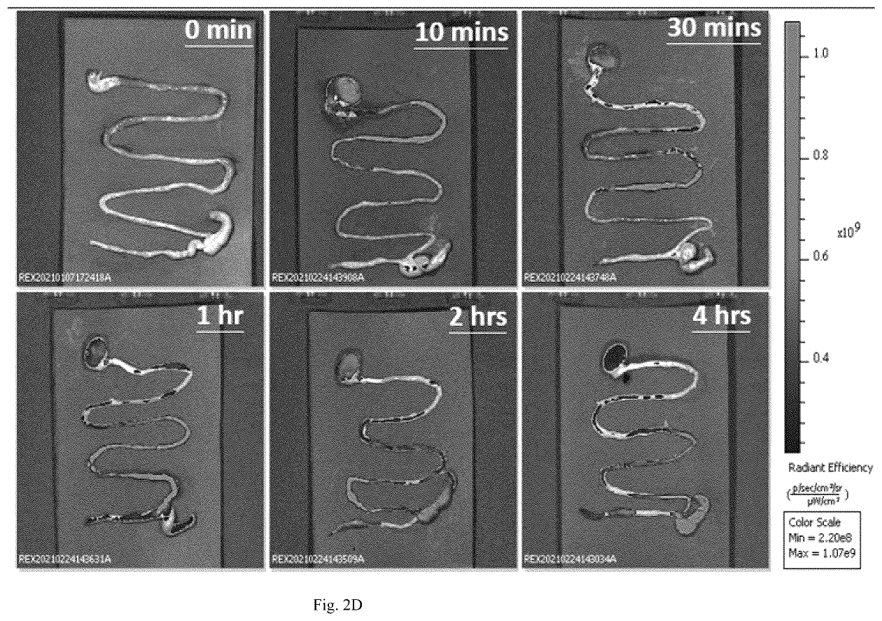

FIG. 2A shows an in vitro mucosal adhesion test. In one embodiment, natural alginate (Alginate, Alg) was labeled with the fluorescent protein FITC to create Alg-FITC (AF). Subsequently, Alg-FITC (AF) was thiolated to obtain TA-FITC (TAF). Small intestinal epithelial cells were then used in a microfluidic model to simulate gastrointestinal adhesion experiments and evaluate the results. The results confirmed that TAF effectively resists the flushing action similar to that of the gastrointestinal tract, thereby increasing the residence time of TAF in the gastrointestinal tract.

As shown in FIG. 2A, fluorescein isothiocyanate (FITC) was used as a commonly used fluorescent labeling method, wherein rat small intestinal crypt epithelial cells (IEC-6) were cultured on μ-slides and coated with alginate-FITC (AF). Thiolated alginate (TA) and fluorescein isothiocyanate (FITC) formed TA-FITC (TAF). The cells were then subjected to constant flow stress using culture medium. Blue color represented nuclear counterstaining, and green color represented FITC fluorescence, which indicated the retention of AF and TAF. After washing with culture medium, the μ-slides were observed under a fluorescence microscope. The relative intensity of FITC was quantified using ImageJ software to evaluate the remaining amount of AF or TAF on the slides and assess the adhesive capacity of thiolated alginate (TA) in vitro. One-way analysis of variance (ANOVA) was performed (n=3, ***p<0.001). FIG. 2A demonstrates an in vitro mucosal adhesion test that seeded μ-slides with small intestinal epithelial cells (IEC-6 cells) for an in vitro gastrointestinal test to evaluate the mucosal adhesion properties of alginate and thiolated alginate (TA) materials. They were covalently labeled with FITC as alginate-FITC (AF) and TA-FITC (TAF), respectively, and dissolved in the culture medium. Under microscopic observation, the materials and cell nuclei were represented by blue and green fluorescence, respectively. The cell numbers in both the AF and TAF groups didn't significantly decrease after washing with the culture medium, indicating that the shear stress at this flow rate didn't cause significant damage to the cells. Although the AF and TAF groups exhibited similar fluorescence signals before refinement, after 2 hours, the signal in the alginate-FITC (AF) group noticeably decreased. On the other hand, the fluorescence signal in the TA-FITC (TAF) group remained strong after steady medium flow.

In one embodiment, FIG. 2B shows an in vitro barrier function test. The thiolated polymer of the present invention is a type of mucosal adhesive polymer. The schematic diagram illustrates the barrier function test in an improved Franz diffusion cell (FIG. 2B). The Franz diffusion cell includes a donor chamber 41, a material layer 42, a mucin layer 43, a cellulose membrane 44, a receiving chamber 45, and a glucose meter 46. Additionally, in one embodiment, thiolated alginate (TA) was used in the improved Franz diffusion cell as the nutritional barrier in the small intestine. One-way analysis of variance (ANOVA) with multiple comparisons was performed (n=6 per group, **p<0.01 compared to the control). The barrier function of thiolated alginate (TA) is evaluated in the simulated intestinal mucosal layer using the Franz diffusion cell, with filter paper coated with mucin simulating the gastrointestinal microenvironment. After a ten-minute barrier test, the glucose level in the thiolated alginate (TA) group was 42.8% lower than that in the control group. In contrast, alginate only reduced glucose permeation by 10.7% compared to the control group. There was a significant difference between the TA and alginate groups. The results of the in vitro barrier function test indicate that thiolated alginate (TA) has better glucose barrier function than alginate.

In the animal intestinal mucosal adhesion test shown in FIG. 2C for the alginate-FITC (AF) group and FIG. 2D for the thiolated alginate-FITC (TAF) group, the materials containing fluorescent proteins were administered to mice via oral gavage. The mice were sacrificed at different time points, and the gastrointestinal tract was extracted and imaged using the IVIS (Luminar II in vivo imaging system). As shown in FIGS. 2C and 2D, after 4 hours (240 minutes) of oral gavage, the residual fluorescent signal in the alginate-FITC (AF) group was significantly lower than that in the thiolated alginate-FITC (TAF) group. These results indicate that the present invention, thiolated alginate (TA), has a longer retention time in the animal body.

As shown in FIGS. 2C and 2D, an in vivo mucosal adhesion test was conducted using the IVIS in accordance with one embodiment. Six male mice (8 weeks old) were used to verify the adhesion properties of AF and TAF on the small intestinal mucosa. After feeding the fluorescent materials, the gastrointestinal tract was collected at 0, 10, 30, 60, 120, and 240 minutes. Mice were sacrificed at different time intervals after oral administration, and the gastrointestinal tract was extracted. Imaging of the gastrointestinal tract was performed using the 3D in vivo bioluminescent imaging system, IVIS. Gastrointestinal tract samples were collected from the stomach to the colon at each time point, and in vivo analysis was conducted using IVIS. The analysis displayed an intensity reduction from yellow to red through color imaging, indicating the fluorescence signal in the gastrointestinal tract from the stomach to the colon. In the presence of fluorescent materials for ten minutes, the distribution of fluorescence between the two materials was similar. After one hour, the fluorescence intensity signals of the two materials began to differ. When observing the autofocus signal for up to four hours, the intensity significantly decreased. In comparison, as shown in FIG. 2D, thiolated alginate-FITC (TAF) shows a stronger fluorescence residue.

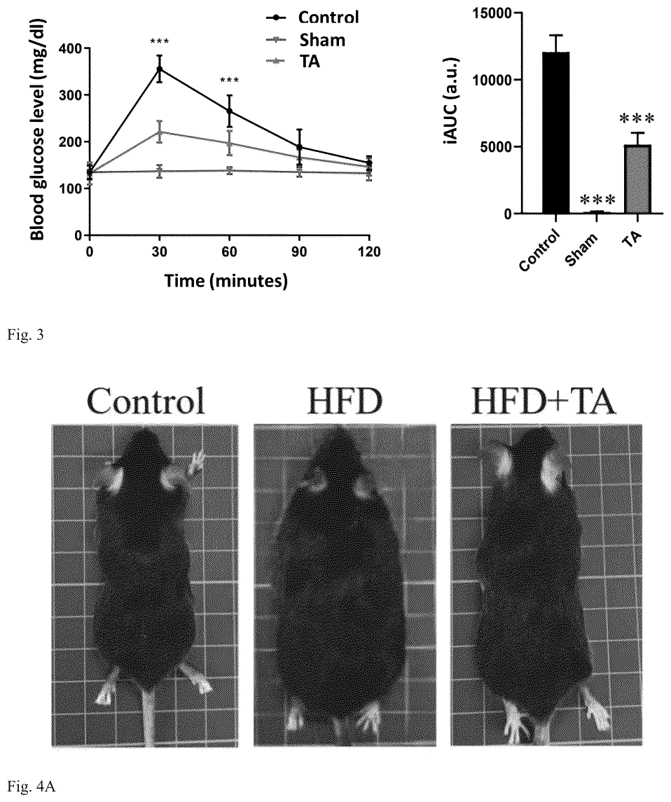

In one embodiment, FIG. 3 evaluates in vivo mucosal adhesion and the oral glucose tolerance test of thiolated alginate (TA). In the curve of the oral glucose tolerance test (OGTT), the following groups were included: (1) The control group, which served as the reference group and was tested with a glucose solution combined with phosphate-buffered saline (PBS). (2) The TA group, which was tested with a glucose solution combined with thiolated alginate (TA). (3) The PBS group, the sham group, which received only phosphate-buffered saline (PBS). One-way analysis of variance (ANOVA) with multiple comparisons was performed (n=10. ***p<0.001). As shown in FIG. 3, the incremental area under the curve (iAUC) was measured. One-way analysis of variance (ANOVA) with multiple comparisons was performed (n=10. ***p<0.001 compared to the control). The results indicate that mice administered with thiolated alginate (TA) effectively reduce the rise in blood glucose levels caused by glucose feeding.

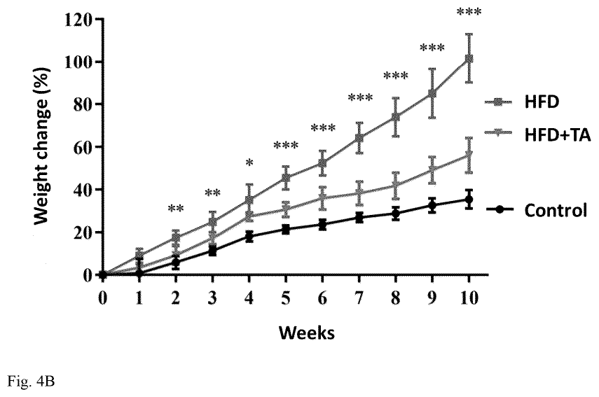

In one embodiment, FIGS. 4A and 4B show the long-term efficacy test of thiolated alginate (TA) in animals. To establish an obesity mouse model, a high-fat diet (HFD) was administered continuously for six weeks. Simultaneously, the mice were orally fed the TA material (HFD+TA) daily to observe the effect of the TA material on body weight control. After ten weeks, the TA group showed a 44.85% reduction in weight gain compared to the high-fat diet group (HFD). As shown in FIGS. 4A and 4B, it demonstrates that long-term administration of thiolated alginate (TA) has a sustained effect on weight reduction.

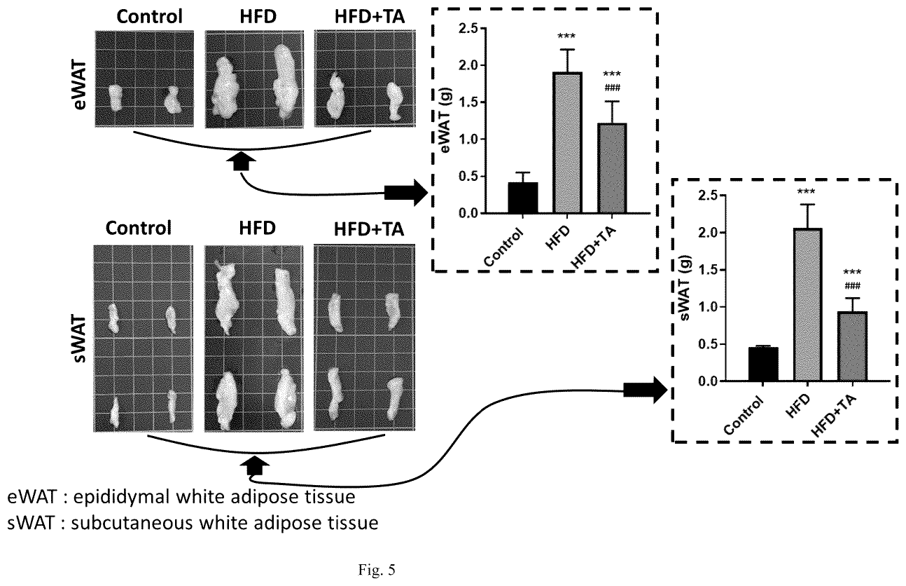

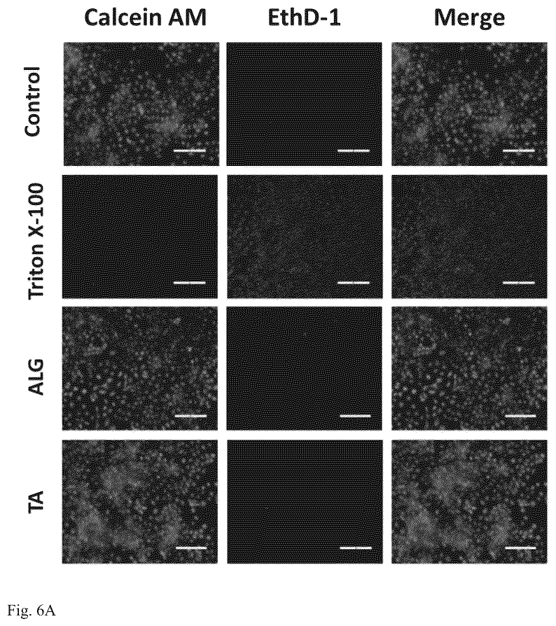

In one embodiment, FIG. 5 shows the weight change test of adipose tissue. The epididymal white adipose tissue and the subcutaneous white adipose tissue of the mice were fed a high-fat diet (HFD) daily in the HFD group, while in the HFD+TA group, the mice were orally fed thiolated alginate (TA) in addition to the high-fat diet (HFD). As shown in FIG. 5, it demonstrates the sustained effect of thiolated alginate (TA) in reducing epididymal white adipose tissue and subcutaneous white adipose tissue in mice.

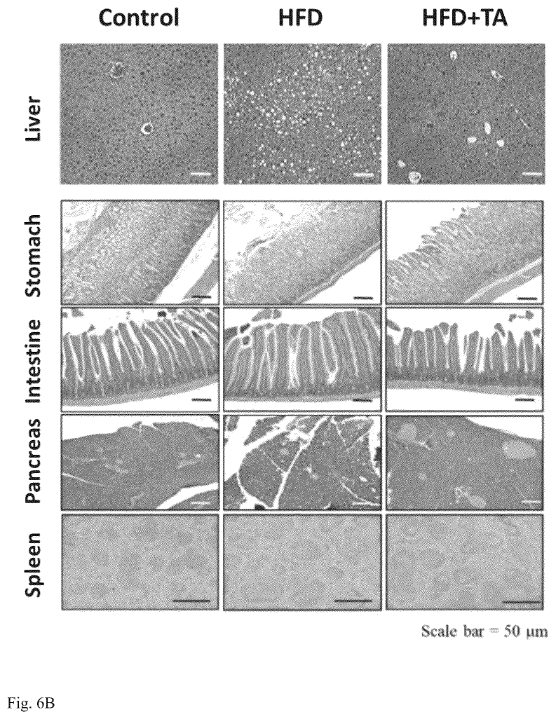

In one embodiment, FIG. 6A shows that a live/dead cell viability assay was conducted to evaluate whether the thiolated alginate (TA) material exhibits significant cytotoxicity towards the L929 cell line. In FIG. 6A, the results of the live/dead cell viability assay were compared to the control group. In contrast to the control group, the experimental group not only showed no significant morphological changes but also exhibited better cell viability. Therefore, the thiolated alginate (TA) material did not demonstrate cytotoxicity and exhibited good biocompatibility. As shown in FIG. 6A, the live/dead staining assessed the cell toxicity of alginate and thiolated alginate (TA), where green color indicates live cells stained with calcein AM, and red color indicates dead cells stained with ethidium homodimer-1 (EthD-1). The scale bar represents 100 μm.

In one embodiment, FIG. 6B uses hematoxylin and eosin stain (H&E stain), one of the most commonly used staining methods in histology. Due to the barrier properties of TA, it can inhibit excessive fat intake, thereby reducing lipid deposition in the viscera. H&E staining is an effective method for histological examination of liver fat accumulation. In comparison to the HFD group, where there was a significant amount of lipid accumulation in the liver slides, the liver slides from the HFD+TA group resembled those of the control group, showing no significant lipid accumulation. Despite the HFD. TA was able to significantly reduce hepatic steatosis, and no differences were observed in H&E staining of other organs such as the stomach, small intestine, and pancreas.

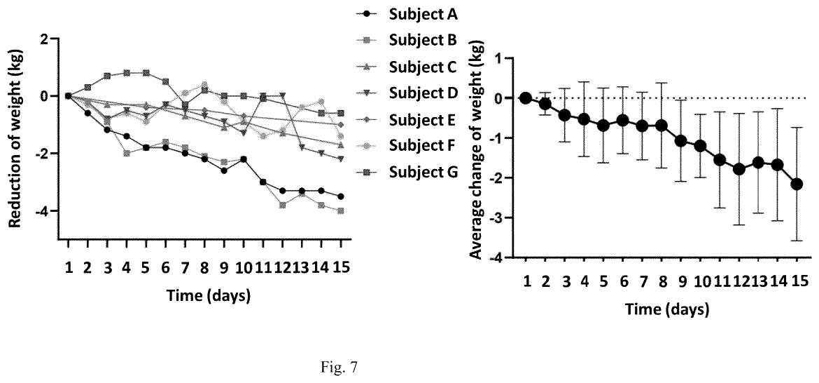

In one embodiment, FIG. 7 shows the reduction in individual weight and average weight for the majority of the subjects. The present invention, thiolated alginate (TA), significantly decreased individual weight and average weight.

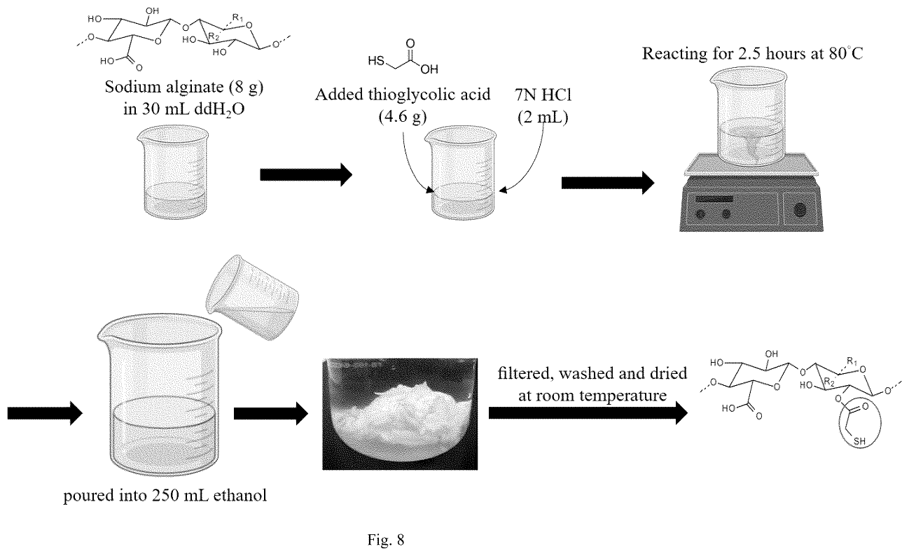

In one embodiment, FIG. 8 shows the preparation of the thiolated alginate (TA) that combines sodium alginate and thiol groups with the covalent bonds by forming ester bonds through the dehydration of both the COOH group and the OH group, which conjugates the thiolglycolic acid (TGA) with the sodium alginate molecule. Dissolve 8 g of sodium alginate in 120 mL of distilled water (ddH₂O) with a molar ratio of 2:1, then use magnetic stirring under acidic conditions (pH 2) at 70-90° C. for 2 hours, along with 1.72 g of thiolglycolic acid (TGA). After the sodium alginate and thiolglycolic acid (TGA) are mixed, the mixture is poured into 300 ml of 95% ethanol to collect the precipitate of thiolated alginate (TA). The collected TA is then freeze-dried and stored at 4° C. for future use.

In one embodiment, alginate-FITC (AF) and thiolated alginate-FITC (TAF) were prepared by labeling alginate and thiolated alginate with FITC, respectively, in mucosal adhesion tests observed under a fluorescence microscope. Dissolve 0.12 g of sodium alginate in 12 mL of sodium acetate buffer (pH 4.9), mix with 50 mg of EDC and 30 mg of NHS, and use magnetic stirring for 30 minutes. NHS refers to N-hydroxysuccinimide. Sodium bicarbonate and 1-ethyl-3-(3-dimethylaminopropyl) carbodiimide (EDC) are added, along with 60 mg of 1,6-diaminohexane, and the reaction is carried out for 4 hours to produce alginateamine. Collect alginate-amine from the precipitate and wash it three times with 95% ethanol. Dissolve the prepared alginateamine in 30 mL of sodium bicarbonate solution (pH 9.0), then add 1 mg of FITC and allow the reaction to proceed for 4 hours. Alginate-FITC (AF) precipitates from 100 mL of 95% ethanol. Freeze-dry and store the obtained product at 4° C. for future use.

The above description and explanations are provided as a description of the preferred examples of the present disclosure. Those skilled in the art would recognize and make modifications based on the scope of the appended claims and the above description. However, these modifications should still fall within the scope of the appended claims for the creative spirit of the present disclosure.

1. A thiolated polymer gel with intestinal absorption barrier, comprising a natural macromolecule (polymer) that has been modified through thiolation and uses thereof creating intestinal absorption barriers, aiding in weight loss, managing diabetes, and addressing metabolic syndrome as a thiolated polymer gel.

2. The thiolated polymer gel with intestinal absorption barrier according to claim 1, comprising:
dissolving a sodium alginate in water to obtain a sodium alginate solution,
mixing a thioglycolic acid (TGA) with the sodium alginate solution under acidic condition at pH 2 and at a temperature range of 70-90° C.,
and pouring the mixture into 95% ethanol to obtain a mixture;
wherein the thiolated alginate (TA) is an alginate modified with the thiol groups, and it forms disulfide bonds in the small intestine, allowing the thiolated alginate (TA) to stay on the mucosal layer of the gastrointestinal system for an extended period, forming a temporary physical absorption barrier between the gastrointestinal system and food nutrients to reduce the absorption of food nutrients in the gastrointestinal system.

3. The thiolated polymer gel with intestinal absorption barrier according to claim 2, wherein the thiolated alginate (TA) is freeze-dried and stored at 4° C. for future use.

4. A method of using the thiolated polymer gel with intestinal absorption barrier, comprising:
administering a reactant carrier that contains the thiolated polymer gel with intestinal absorption barrier, wherein the reactant carrier is formed by the thiolated alginate (TA) as claimed in claim 1 and can be in at least one of the following forms: chewing gum, granulated candy, granulated chocolate, granulated biscuit, pellet, tablet, powder, or capsule.

5. A method of using the thiolated polymer gel with intestinal absorption barrier, comprising:
administering the thiolated alginate as claimed in claim 1 through at least one implementation selected from endoscopic examination, needle, brush, puncture device, tube, straw, or spray.
